(19) United States
(12) Patent Application Publication
(10) Pub. No.: US 2023/0220644 A1
(43) Pub. Date: Jul. 13, 2023

(54) A MEASURING DEVICE AND METHOD OF USE THEREOF

(71) Applicant: Yvo KEULEMANS, Manly West (AU)

(72) Inventor: Yvo KEULEMANS, Manly West (AU)

(21) Appl. No.: 18/001,627

(22) PCT Filed: Jun. 18, 2021
(86) PCT No.: PCT/AU2021/050630

§ 371 Date: Dec. 13, 2022

(30) Foreign Application Priority Data
Jun. 18, 2020 (AU) 2020902022

(51) Int. Cl. E02D 1/02

(52) U.S. Cl. CPC E02D 1/027; E02D 1/022

(57) ABSTRACT

The present invention concerns a device and method for selectively or simultaneously measuring shear strength and pore water pressure of a soil in the field. The device includes a rod adapted to be at least partially inserted into the soil and rotated. The rod has a soil engaging portion and an opposed coupling portion configured to be coupled to a torque applying machine or device. The device further includes at least one vane blade extending at least partially along and from the soil engaging portion of the rod for shearing the soil when rotated together with the rod. At plurality of pore water pressure sensors are operatively associated with at least one of the soil engaging portion and the at least one vane blade. The sensors are configured to sense pressure indicative of the pore water pressure of the soil while the at least one vane blade shears the soil.

### TECHNICAL FIELD

The present invention relates to a device, system and method for use in the field of geotechnical engineering. In particular, the present invention concerns a device, system and method for selectively or simultaneously measuring shear strength and pore water pressure of a soil in the field.

### BACKGROUND

The field vane shear test is a test for determining the shear strength of a soil in the field by measuring the torque required to cause a vane of cruciform section to shear the soil. The test is suitable for use with very soft to firm non-fissured clays, silts or other saturated fine-grained geomaterials, such as, e.g., mine tailings and organic muck.

Generally, the test is a measure of a soil's strength while the soil remains fully saturated with water. The resultant shear strength reading is called the “undrained shear strength” of the soil.

Importantly, the saturation, or moisture content, of the soil can greatly affect the accuracy of the field vane shear test.

For example, the vane shear test is typically not applicable to unsaturated soils or in non-plastic silts, sands, gravels or other high permeability soils wherein the resulting drained shear strength does not conform to Australian or international standards.

Further, and in other examples, the water between soil particles may become excessively compressed during a field vane shear test and may result in an underestimation or overestimation of the shear strength of the soil.

Generally speaking, a problem with the current Australian and international standards that regulate the field vane shear test is that there is no way to determine the interplay between the pore water pressure and the shear strength of soil while a test is being performed. Thus, there is no way to determine whether a field vane shear test complies with the respective standards.

As demonstrated at the Fundão dam, an iron ore tailings dam in Mariana, Minas Gerais, Brazil, on 5 Nov. 2016, an underestimation or overestimation of the shear strength of an earthen structure, can result in catastrophic environmental disasters when the earthen structure fails due to a loss of stability.

Vanes have been previously developed that have the capability to measure pore water pressure. However, the present inventor has found that such vanes are typically poorly designed to provide an accurate pore water measurement.

For example, U.S. Pat. No. 5,109,702 discloses a vane incorporating a pore water pressure transducer for determining a liquefaction potential of cohesionless soils when determining the shear strength of the soil. However, the present inventor has found that the disclosed vane includes a number of limitations that detract from the working of the vane, including reliance on a removable cap located at a bottom of the vane for securely sealing a duct passageway system, a propensity for the pore water pressure inlets to clog and poor placement of the pore water pressure inlets for an accurate determination of the pore water pressure.

Chinese Patent Publication No. 102943460 A similarly discloses a vane incorporating a pore water pressure transducer for determining pore water pressure of a soil. However, like with U.S. Pat. No. 5,109,702, the present inventor has found that the disclosed vane has similar deficiencies that detract from the working of the vane. For example, the disclosed vane also relies on a removable water plug located at a bottom of the vane for securely sealing a duct passageway system and has pore water pressure inlets that are poorly placed and prone to clogging to otherwise provide an accurate determination of the pore water pressure.

It will be clearly understood that, if a prior art publication is referred to herein, this reference does not constitute an admission that the publication forms part of the common general knowledge in the art in Australia or in any other country.

### SUMMARY OF INVENTION

Embodiments of the present invention provide a device, system and method for selective or simultaneous measurement of shear strength and pore water pressure, which may at least partially address one or more of the problems or deficiencies mentioned above or which may provide the public with a useful or commercial choice.

According to a first aspect of the present invention, there is provided a device for selectively or simultaneously measuring shear strength and pore water pressure of a soil in the field, said device including:

a rod adapted to be at least partially inserted into the soil and rotated, said rod having a soil engaging portion and an opposed coupling portion configured to be coupled to a torque applying machine or device;

at least one vane blade extending at least partially along and from the soil engaging portion of the rod for shearing the soil when rotated together with the rod; and

at least one pressure sensor operatively associated with at least one of the soil engaging portion and the at least one vane blade, and configured to sense pressure indicative of the pore water pressure of the soil while the at least one vane blade shears the soil.

According to a second aspect of the present invention, there is provided a device for selectively or simultaneously measuring shear strength and pore water pressure of a soil in the field, said device including:

a rod adapted to be at least partially inserted into the soil and rotated, said rod having a soil engaging portion and an opposed coupling portion for coupling to a toque applying machine or device;

at least one vane blade extending at least partially along and from the soil engaging portion of the rod for shearing the soil when rotated together with the rod, said at least one vane blade having a leading face, an opposed trailing face, an upper edge, an opposed lower edge and an outer side edge; and

a plurality of pore water pressure sensors operatively associated with two or more of the soil engaging portion, the leading face, the trailing face and the outer side edge, said sensors configured to collectively sense pressure indicative of the pore water pressure of the soil while the at least one vane blade shears the soil.

According to a third aspect of the present invention, there is provided a system for selectively or simultaneously measuring shear strength and pore water pressure of a soil in the field, said system including:

a device in accordance with the first or second aspects; and

a torque applying machine having a controller coupled to the coupling portion of the rod of the device for rotating the rod, said controller configured to control rotation of the rod at a desired rate of rotation.

Advantageously, the device and system of the present invention enables a field vane shear test to be accurately performed in compliance with both Australian and international standards. By measuring the pore water pressure while determining the shear strength, the device enables the effect of the pore water pressure to be observed and measured during the field vane shear test. Moreover, the device enables the effect of the pore water pressure to be accounted for when determining the undrained shear strength of the soil thereby providing a more accurate measurement of the shear strength of the soil. Embodiments of the device of the present invention will greatly reduce the likelihood of an under or over estimation of the undrained shear strength of a soil layer of interest. Further, embodiments of the present invention by including multiple pore water pressure sensors at differing locations advantageously provide a much more accurate reading of the porewater pressure of the soil than conventional vanes.

As indicated, the device is for selectively or simultaneously measuring shear strength and pore water pressure of a soil in the field. Generally, the soil may form part of an earthen substrate. For example, the earthen substrate may include a portion of land, an embankment, a levee, a transportation embankment, an embankment dam, an earthen dam wall (e.g., a tailings dam wall) or reclaimed land alongside a body of water, such as, e.g., a riverbank or beach.

Pore water pressure may refer to the pressure of groundwater held within soil gaps between particles.

Groundwater may refer to water present beneath or within the earthen substrate in soil pore spaces and fractures and void spaces of rock formations.

The soil may refer to any non-fissured clay, silt or other saturated fine-grained geomaterials, such as, e.g., mine tailings, organic muck, slimes, leach residues, slickens, dredge spoils and other like material and/or materials.

The rod may be of any suitable size, shape and construction and formed from any suitable material or materials to be at least partially inserted into the soil and rotated.

For example, the rod may be formed from plastic, carbon fibre, carbon composite or metal material or materials, preferably steel, more preferably stainless steel.

The rod may include a pair of opposed ends and may extend longitudinally between the opposed ends in a linear direction. The opposed ends may include a soil engaging end and an opposed torque applying machine or device engaging end.

The rod includes a soil engaging portion located at or near the soil engaging end and coupling portion for coupling to a torque applying machine or device located at or near the torque applying machine or device engaging end.

The coupling portion may couple to a torque applying machine or device in any suitable way for torque to be transmitted for rotation of the rod. For example, the coupling portion may couple directly, or indirectly via a torque rod, with the torque applying machine or device.

The torque applying machine or device may include an actuating mechanism for rotation of the rod in a clockwise or anti-clockwise direction. Any suitable type of actuating mechanism may be used. The actuating mechanism may be manually actuated or by using a drive motor, preferably the latter.

If manually actuated, the torque applying machine or device may include an operable handle or crank associated with one or more gears or cog wheels configured to mesh with one another when the handle or crank is turned and transmit torque to the coupling portion of the rod.

If using a drive motor, the torque applying machine or device may include an electric motor or combustion engine operatively associated with the coupling portion of the rod for rotating the rod. A shaft of the drive motor may be interconnected with the coupling portion by one or more gears, chains, pulleys, belts or other linkages, and may also, optionally use one or more clutches. In some embodiments, the coupling portion of the rod may be directly coupled to the shaft of the drive motor. In preferred embodiments, the drive motor may be interconnected with the coupling portion of the rod by one or more gears capable of driving rotation of the shaft at different rates of rotations with anywhere up to 200 Nm of torque.

The torque applying machine or device may enable rotation of the rod and the at least one vane blade at a desired rate of rotation.

For example, in manually actuated embodiments, the one or more gears or cog wheels may regulate or control rotation of the rod to assist an operator in rotating the device at a desired rate of rotation.

Likewise, in drive actuated embodiments, the machine or device may be configurable to rotate the rod and the at least one vane blade at a desired rate of rotation. Typically, in such embodiments, the machine or device may include a controller for controlling rotation of the rod.

The controller may preferably be in the form of a computing device including one or more processors and a memory. In such embodiments, the controller may include software configured to be run on the computing device for controlling operation of the machine or device, such as, e.g., a rate of rotation, an angle of rotation, an amount of torque applied. The software may be interactive, preferably such that an operator may interact and control operation of the machine or device.

In such embodiments, the device of the present invention may rotated at a rate of rotation of about 0.05° per second, about 0.10° per second, about 0.15° per second, about 0.20° per second, about 0.25° per second, about 0.30° per second, about 0.35° per second, about 0.40° per second, about 0.45° per second, about 0.50° per second, about 0.55° per second, about 0.60° per second, about 0.65° per second, about 0.70° per second, about 0.75° per second, about 0.80° per second, about 0.85° per second, about 0.90° per second, about 0.95° per second, about 1.00° per second, preferably about 0.10° per second.

The coupling portion and the torque applying machine or device may generally be connected together by a connecting mechanism or part of a connecting mechanism. For example, a first part of the connecting mechanism associated with the coupling portion may mate or engage with a second part of the connecting mechanism associated with the torque applying machine or device.

The connecting mechanism may include a threaded connection, an interference fit (snap fit) connection or a bayonet-type connection, for example.

The connecting mechanism may involve a male formation engaging a female formation. For example, in some embodiments, the connecting mechanism may include the coupling portion having a male formation that engages with, or is at least partially inserted into, a female formation associated with the torque applying machine or device. In other embodiments, the coupling portion may include the female formation that engages with, or at least partially receives, the male formation associated with the torque applying machine or device.

In preferred embodiments, the coupling portion may be configured to be at least partially inserted into a casing associated with the torque applying machine or device or a torque rod associated with the torque applying machine or device. The casing may be configured transmit axial rotation and torque to the rod without the rod tightening or twisting relative to the casing or a torque rod.

In some embodiments, the casing may include a clamping mechanism for clamping at least a portion of the coupling portion of the rod.

In other embodiments, the coupling portion may include a shaped head and the casing may include a complementary-shaped socket configured to at least partially receive the shaped head.

The rod may be of any suitable length as defined between the opposed ends. For example, the rod may have a length of about 400 mm, about 450 mm, about 500 mm, about 550 mm, about 600 mm, about 650 mm, about 700 mm, about 750 mm, about 800 mm, about 850 mm, about 900 mm, about 950 mm, about 1,000 mm, about 1,050 mm, about 1,100 mm, about 1,150 mm, about 1,200 mm, about 1,250 mm, about 1,300 mm, about 1,350 mm, about 1,400 mm, about 1,450 mm, about 1,500 mm, or greater.

Likewise, the rod may be of any suitable diameter. For example, the rod may have a diameter of about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, about 26 mm, about 27 mm, about 28 mm, about 29 mm, about 30 mm, about 31 mm, about 32 mm, about 33 mm, about 34 mm, about 35 mm, about 36 mm, about 37 mm, about 38 mm, about 39 mm or even about 40 mm.

As indicated, the device includes at least one vane blade extending at least partially along and from the soil engaging portion.

The at least one vane blade may be of any suitable size, shape and construction for shearing the soil when the rod is rotated.

Generally, the at least one vane blade may be formed from plastic, carbon fibre, carbon composite or metal material or materials, preferably steel, more preferably stainless steel.

The at least one vane blade may have a polygonal shape. For example, the blade may be in the shape of a triangle, rectangle, pentagon, hexagon or octagon. In preferred embodiments, the blade may be substantially rectangular in shape.

The at least one vane blade may have a pair of opposed side surfaces extending substantially parallel to one another. The opposed side surfaces may include a leading face facing a direction of travel when rotated in a soil and an opposed trailing face. The opposed side surfaces may be interconnected by opposing edges, including an upper edge, an opposed lower edge, an inner side edge and an opposed outer side edge.

The at least one vane blade may be joined or connected to the rod in any suitable way. For example, the at least one vane blade may be permanently or releasably joined or connected to the rod, preferably the former.

In some embodiments, the rod and the at least one vane blade may be of unitary construction.

In other embodiments, the rod and the at least one vane blade may be joined together using conventional welding techniques.

In yet other embodiments, the rod and the at least one vane blade may be fastened together by one or more mechanical fasteners.

In some embodiments, the blade may extend at an angle from the soil engaging portion of the rod. For example, the blade may be angled towards or away from a direction of rotation.

In other preferred embodiments, the blade may extend from the rod in a direction perpendicular to the longitudinal axis of the rod.

The blade may extend any suitable distance outwardly and partially along the soil engaging portion of the rod.

The distance that the blade extends outwardly from the soil engaging portion of the rod may correspond to a width of the blade. The distance that the blade extends at least partially along the soil engaging portion of the rod may correspond to a height of the blade.

For example, the blade may have a width, as measured between the inner and outer side edges, of about 20 mm, about 25 mm, about 30 mm, about 35 mm, about 40 mm, about 45 mm, about 50 mm, about 55 mm, about 60 mm, about 65 mm, about 70 mm, about 75 mm, about 80 mm, about 85 mm, about 90 mm, about 95 mm, about 100 mm, about 150 mm, about 200 mm, about 250 mm, about 300 mm, about 350 mm, about 400 mm, about 450 mm, about 500 mm or more.

Likewise, the blade may have a height, as measured between the lower and upper edges, of about 40 mm, about 50 mm, about 60 mm, about 70 mm, about 80 mm, about 90 mm, about 100 mm, about 150 mm, about 200 mm, about 250 mm, about 300 mm, about 350 mm, about 400 mm, about 450 mm, about 500 mm, 550 mm, about 600 mm, about 650 mm, about 700 mm, about 750 mm, about 800 mm, about 850 mm, about 900 mm, about 950 mm, about 1,000 mm or more.

In some embodiments, the blade height may be greater than the blade width. In other embodiments, the blade height may be less than the blade width.

In some embodiments, the blade height and width may be directly proportional to one another. For example, in some such embodiments, the blade height may be about one and a half-times, about two-times, about three-times, about four-times, or even about five-times the width of the blade. In other such embodiments, the blade width may be about one and a half-times, about two-times, about three-times, about four-times, or even about five-times the height of the blade.

In some embodiments, the blade height may be about four-times the width of the blade. In other embodiments in which the device may include a diametrically opposed pair of blades extending from opposite sides of the soil engaging portion, the blade height may be about two-times the diameter of the pair of blades.

The blade may also be of any suitable thickness, as measured between the opposed surfaces. For example, the blade may have a thickness of about 1.0 mm, about 1.5 mm, about 2.0 mm, about 2.5 mm, about 3.0 mm, about 3.5 mm, about 4.0 mm, about 4.5 mm, or even about 5.0 mm or more. Typically, the blade may have a thickness ranging between about 1.5 mm and about 3.5 mm.

In some embodiments, the size of the blade may be calculated according to the standards to have a pre-determined surface area ratio relative to a remainder of the device. For example, surface area ratio of the blade may be calculated according to the following equation:

$$Area ratio = \frac{8 t ( D - d ) + π {d}^{2}}{π {D}^{2}} \times 100$$

where:

t=the thickness of the vane blade, in millimetres

D=the blade width, in millimetres

d=diameter of the rod, in millimetres.

In some embodiments, the at least one vane blade may include rounded, sharpened and/or tapered edges. For example, one or more of the upper edge and the outer side edge may be tapered to be thinner to reduce disturbance from insertion of the device into the soil. Likewise, in some such embodiments, the lower edge of the at least one vane blade may be sharpened to facilitate penetration of the device into the soil.

In preferred embodiments, the device may include more than one vane blade. For example, the device may include two, three, four, five or even six vane blades, preferably equally spaced about the soil engaging portion of the rod.

In some embodiments, the blades may be arranged in diametrically opposed pairs about the soil engaging portion of the rod.

As indicated, the device includes at least one pressure sensor operatively associated with the soil engaging portion and/or the at least one vane blade. The at least one pressure sensor may be of any suitable size, shape and form.

Generally, the at least one pressure sensor may be provided in a casing including at least one groundwater inlet port, and the at least one pressure sensor may measure the pressure of the groundwater at the groundwater inlet port, said pressure being indicative of the pore water pressure.

Typically, the at least one pressure sensor may be discretely located on a portion of the soil engaging portion and/or the at least one vane blade. Advantageously, by being discretely located, the pressure sensor does not need to rely on a duct passageway system interconnecting multiple pore water pressure inlets and therefore does not require a plug or cap located at the bottom of the device for draining and/or filing the duct passageway with water prior to and after use.

The at least one pressure sensor may be an electronic pressure sensor, preferably a piezoelectric sensor.

In some embodiments, the pressure sensor may measure gauge pressure. For example, the pressure sensor may measure the pressure relative to atmospheric pressure.

In other embodiments, the pressure sensor may measure absolute pressure. For example, the pressure sensor may measure the pressure relative to a vacuum.

In yet other embodiments, the pressure sensor may measure the pressure relative to a reference fluid of known pressure or density. For example, the pressure sensor may measure a pressure differential between the groundwater and the reference fluid.

Any suitable fluid may be used as a reference fluid provided the fluid has a density amenable to the sensitivity of the pressure sensor for measuring a pressure difference between the reference fluid and the groundwater. The reference fluid may be a gas or a liquid, preferably a gas, such as, e.g., air.

The casing may be of any suitable size and shape to enable a flow of groundwater into the inlet port and pressure indicative of the pore water pressure to be accurately measured.

The groundwater inlet port of the casing may include an opening of suitable size to allow the passage of groundwater. For example, the opening may have a diameter or maximum width of at about 1 mm, at least about 2 mm, at least about 3 mm, at least about 4 mm, at least about 5 mm, at least about 6 mm, at least about 7 mm, at least about 8 mm, at least about 9 mm, at least about 10 mm, at least about 11 mm, at least about 12 mm, at least about 13 mm, at least about 14 mm, at least about 15 mm, at least about 16 mm, at least about 17 mm, at least about 18 mm, at least about 19 mm or even at least about 20 mm.

In some embodiments, the groundwater inlet port may further include a cavity in fluid communication with the opening for receiving groundwater that flows through the opening.

In some embodiments, the groundwater inlet port of the casing may further include a screen or filter to prevent the passage of materials other than groundwater into the opening and/or cavity of the casing. The screen or filter may be sufficiently fine to prevent the passage of soil materials but allow the passage of the groundwater. For example, the screen or filter may have a pore size between about 2 μm and about 20 μm.

The screen or filter may be removable and/or replaceable so that at least the screen or filter may be cleaned and re-fitted or replaced between measurements or as required.

In some embodiments, the groundwater inlet port may be filled with a fluid other than water and pressure indicative of the pore water pressure may be measured based on the pressure of the groundwater on the fluid in the cavity. The fluid may have a viscosity greater than water, preferably the fluid may be selected from any one of glycerin, silicone oil and grease.

The fluid may be added and/or removed between measurements or as required.

Advantageously, by filling the groundwater inlet port with a fluid other than water prior to use and determining pore water pressure based on the pressure of the groundwater on the fluid in the inlet port, the inlet port itself is less prone to being clogged with soil debris.

In some embodiments, the casing including the at least one pressure sensor and the groundwater inlet port may be located on the at least one vane blade.

For example, in some such embodiments, the casing may be located on any one of the upper edge, the lower edge and the outer side edge of the vane blade, preferably the outer side edge. In such embodiments, the casing may preferably be received in a cavity defined in the edge such that the groundwater inlet port of the casing lies substantially flush with a remainder of the edge.

In other such embodiments, the casing may be located on any one of the opposed side surfaces of the vane blade. For example, the casing may be located on a leading or trailing face of the vane blade, or both. Again, in such embodiments, the casing may preferably be received in a cavity defined in the side surface or surfaces such that the groundwater inlet port of the casing lies substantially flush with the surface or surfaces.

In other embodiments, the casing including the at least one pressure sensor and the groundwater inlet port may be located on a portion of the soil engaging portion of the rod.

For example, in such embodiments, the casing may be located above or below the at least one vane blade, or in embodiments in which the device includes more than one vane blade, the casing may be located between the vane blades.

In some embodiments, the device may include a plurality of pressure sensors. For example, the device may include at least two, at least three, at least four, at least five, at least six, at least seven or even at least eight pressure sensors. Each sensor may be housed within a casing including at least one groundwater inlet port and may be configured to sense pressure of the groundwater indicative of the pore water pressure.

In such embodiments, the plurality of pressure sensors may be located at various locations on the device.

For example, in some such embodiments, pressure sensors may be located on an outer side edge of each vane blade.

In another such embodiment, at least one pressure sensor may be located on a side surface of each vane blade, preferably on both side surfaces of each vane blade.

In yet other embodiments, the at least one vane blade may include a plurality of pressure sensors. For example, the blade may include sensors on two or more of the soil engaging portion, the leading face, the trailing face and the outer side edge, preferably at least the leading face and the outer side edge. Each sensor may be housed within a casing including at least one groundwater inlet port. Each sensor may be capable of selectively or collectively sensing pressure of the groundwater indicative of the pore water pressure.

In embodiments in which the device includes more than one vane blade, each vane blade may include one or more pressure sensors. Alternately, only some vane blades may include one or more pressure sensors. For example, in a device including at least four vane blades arranged in diametrically opposed pairs, one pair may include one or more pressure sensors on each blade while the other pair may not include any pressure sensors. Conversely, all blades may each include one or more pressure sensors.

The at least one pressure sensor may be electrically connected to a data bus or like connection, for example.

In some embodiments, a dedicated microprocessor or microcomputer, including one or more processors and a memory, may be operatively associated with the at least one pressure sensor for collecting data corresponding to said pressure sensed and transmitting the data to an external device, controller or processing device. The data may be at least partially transmitted via at least one electrical circuit extending along and within the rod, or portions thereof. The at least one electrical circuit may include a data bus, a twisted pair network and/or a fibre optic network, for example. Excitation/operative voltage may be supplied over the circuit (such as POE) or separately.

The microprocessor or microcomputer may convert resistance measurements to pressure reading using any suitable algorithm. For example, the algorithm may be embodied by the equation:

P=m·R−o

where:
P=the pressure
R=the Resistance
m=a constant−the coefficient
o=a constant−the offset (equal to the pressure that would correspond to zero resistance (R₀)).

In some embodiments, the device may further include at least one amplifier for amplifying an output electrical signal from the pressure sensor indicative of the pore water pressure sensed.

The at least one amplifier may be operatively connected to the pressure sensor, and other electronic components of the device via the at least one electrical circuit.

In embodiments in which the pressure sensor may provide an analog output signal, the device may further include an analog-to-digital converter for converting the analog signal to a digital signal. The analog-to-digital converter may communicate a digital signal indicative of the analog output signal from the pressure sensor. The analog-to-digital converter may be operatively connected to the at least one amplifier, if present, the pressure sensor and other electronic components of the device again via the at least one electrical circuit.

In some embodiments, the device may include a communications module for connecting the device to an external device, such as, e.g., an external processing device (e.g., computer, tablet, smart phone, smart watch or PDA), a controller, an external display or a storage device. The device may be connected to the external device in any suitable way.

For example, in some such embodiments, the communication module may be in the form of a port or access point (e.g., a USB or mini-USB port) such that the device may be connected to an external device using a suitable cable.

In other such embodiments, the communications module may be in the form of a wireless communications module, such as, e.g., a wireless network interface controller, such that the device may wirelessly connect to the external device via a wireless network (e.g., Wi-Fi (WLAN) communication, Satellite communication, RF communication, infrared communication or Bluetooth™). In such embodiments, the communications module may include at least one modem, such as, e.g., a cellular or radio modem.

In some embodiments, the device may include a power supply for powering electrical components of the device, including the at least one pressure sensor. The power source may include an on-board power source, such as, e.g., one or more batteries, preferably rechargeable batteries. In other embodiments, the power source may include an external power source, such as, e.g., a power source operatively associated with the torque applying machine or device.

In some embodiments, the device further includes a controller for controlling operation of the device and for receiving data indicative of shear strength, pore water pressure, degrees of rotation, rate of rotation and for remotely controlling operation of the device. The controller may be operatively connected to the device for receiving output data from the device and for controlling operation of the device, or at least controlling aspects of operation of the device. The controller may be wired or wirelessly connected to the device.

The controller may include a computing device, including one or more processors and a memory. In such embodiments, the controller may include software configured to run on the computing device for controlling operation of the device, or at least aspects of operation of the device. The software may be interactive and allow an operator interact with and control operation of the device.

In some such embodiments, the controller may also be configured to control operation of the torque applying machine or device coupled to the device. In other such embodiments, the controller may be configured to interact with the controller previously described for controlling operation of the torque applying machine or device.

In some embodiments, the controller may be a remote controller. The remote controller may be of any suitable size, shape and form.

The remote controller may include one or more keys, buttons and/or switches for an operator to control operation of the device.

In some such embodiments, the remote controller may include at least one display for displaying data transmitted from the device, such as, e.g., pore water pressure, rate and angle or rotation as well as torque.

The remote controller may include a microcomputer, including one or more processors and a memory.

In some embodiments, the remote controller may be in the form of a computing device, such as, e.g., a laptop or desktop. In such embodiments, the device may include software configured to be run on the computing device for controlling operation of the device, or at least aspects of operation of the device. The software may preferably be interactive and allow an operator to interact and control operation of the device.

In some embodiments, the remote controller may be in the form of a mobile computing device, such as, e.g., a smart phone, a tablet or a smart watch. In such embodiments, the remote controller or device may further include software in the form of an application (i.e., an app) configured to be run on the mobile computing device and allow an operator to interact with and control the device, or at least aspects of operation of the device.

In some embodiments, the device may further include a cleaning mechanism for cleaning or flushing the at least one pore water pressure sensor of soil and/or debris.

In such embodiments, each pore water pressure sensor may be operatively associated with a source of fluid for flushing the sensor following or in between measurements, preferably the same fluid as used to fill the cavity and/or the casing containing the sensor (e.g., glycerin, silicone oil and/or grease).

The fluid may be delivered from a fluid source into the cavity and/or the casing of each pressure sensor, preferably via one or more outlets in fluid communication with the cavity and/or the casing and the source.

In some embodiments, the fluid source may be an external source connectable to the one or more outlets via one or more conduits extending at least partially along the rod to the pressure sensors, preferably in an internal arrangement relative to the rod. In such embodiments, the fluid may be dispensed under pressure from the external source, typically via a pump for pumping the fluid from the external source to the pressure sensor.

In other embodiments, the fluid source may be a pressurised container adapted to be dispensed by one or more valves. When the one or more valves are opened, the fluid from the container may be released into the cavity and/or the casing containing the pressure sensor to flush the cavity and/or the casing clean of soil and/or debris. Again, the pressurised container may be connectable to the one or more outlets via one or more conduits extending at least partially along the rod to the pressure sensors, preferably in an internal arrangement relative to the rod.

According to a fourth aspect of the present invention, there is provided a method of selectively or simultaneously measuring shear strength and pore water pressure of a soil in the field, said method including:

providing the device of the first or second aspects or the system of the third aspect; at least partially inserting the soil engaging portion and the at least one vane blade into the soil;

applying torque to the coupling portion to rotate the rod and the at least one vane blade to cause the at least one vane blade to shear the soil; and

monitoring and collecting at least data corresponding to pore water pressure from the at least one pressure sensor simultaneously with said applying.

According to a fifth aspect of the present invention, there is provided a method of selectively or simultaneously measuring shear strength and pore water pressure of a soil in the field, said method including:

at least partially inserting into the soil a rod having at least one vane blade extending from a lower soil engaging end thereof and at least one pressure sensor operatively associated with at least one of the soil engaging end and the at least one vane blade;

applying torque to an opposed upper end of the rod to rotate the rod and the at least one vane blade to cause the at least one vane blade to shear the soil; and

monitoring and collecting at least data corresponding to pore water pressure from the at least one pressure sensor simultaneously with said applying.

The methods of the fourth and/or fifth aspects may include one or more characteristics or features of the device as hereinbefore described.

As indicated, the method may be used for selectively or simultaneously measuring the shear strength and/or the pore water pressure of the soil. However, for the sake of clarity, the pore water pressure is measured while torque is applied to the rod and the at least one vane blade to provide a measurement of the pore water pressure that takes into account the effect of the rotating vane blade on the soil.

The at least partially inserting may include inserting the device or rod into the soil to a layer of interest either directly or via a bore or drill hole. The device or rod is inserted until the at least one vane blade fully penetrates the intact soil layer of interest.

The applying torque includes applying torque until the rod and at least one vane blade rotates at a constant rate. The applying torque is continued until the at least one vane blade causes the soil to shear. Preferably, the applying torque further includes determining the torque required to cause the at least one vane blade to shear the soil. The torque required to cause the at least one vane blade to shear the soil may be noted and recorded. The torque may continue to be applied for any number of rotations or until the shear vane test complies with Australian or international standards.

Simultaneously as the torque is applied, data indicative of the pore water pressure is monitored and collected from the at least one pressure sensor.

The monitoring and collecting of data may occur in real time and continuously while torque is applied to the device or rod.

In some embodiments, said monitoring and collecting may also include monitoring and collecting data corresponding to a degrees of rotation and/or rate of rotation of the rod and the at least one vane blade.

The undrained shear strength of the soil in the layer of interest is then determined based on the maximum torque reached. A microprocessor or microcomputer operatively associated with the device or rod may convert the maximum torque reading to an undrained shear strength value using any suitable algorithm. For example, the algorithm may be embodied by the following equation:

$$S = \frac{{10}^{9} \times 6}{π} \times \frac{T}{{D}^{2} ( 3 H + D )}$$

where:

S=vane shear strength, kilopascals

T=torque to shear the soil, in kilonewton metres

D=overall vane blade width, in millimetres

H=height of vane blade, in millimetres.

The method may further include adjusting the undrained shear strength of the soil determined to account for the pore water pressure of the soil. For example, effect of the pore water pressure may be accounted for and/or subtracted from the torque determined to provide an accurate measurement of the shear strength of the soil.

The method may further include displaying data indicative of the shear strength and the pore water pressure of the soil on a display of a remote controller or external device in communication with the device or rod.

Any of the features described herein can be combined in any combination with any one or more of the other features described herein within the scope of the invention.

The reference to any prior art in this specification is not, and should not be taken as an acknowledgement or any form of suggestion that the prior art forms part of the common general knowledge.

### BRIEF DESCRIPTION OF DRAWINGS

Preferred features, embodiments and variations of the invention may be discerned from the following Detailed Description which provides sufficient information for those skilled in the art to perform the invention. The Detailed Description is not to be regarded as limiting the scope of the preceding Summary of Invention in any way. The Detailed Description will make reference to a number of drawings as follows:

FIG. 1 is a photograph showing an upper perspective view of a device according to an embodiment of the present invention;

FIG. 2 is an upper perspective view showing the device according to another embodiment of the present invention;

FIG. 3 is an upper perspective view showing the device according to yet another embodiment of the present invention;

FIG. 4 is an upper perspective view showing the device according to a further embodiment of the present invention;

FIG. 5 is a flowchart showing steps in a method of using the device as shown in FIGS. 1 to 4 according to an embodiment of the present invention;

FIG. 6 is a graph plotting pore water pressure against time during a field vane shear test using the device as shown in FIG. 1; and

FIG. 7 is another graph plotting pore water pressure against time during a field vane shear test using the device as shown in FIG. 1.

### DETAILED DESCRIPTION

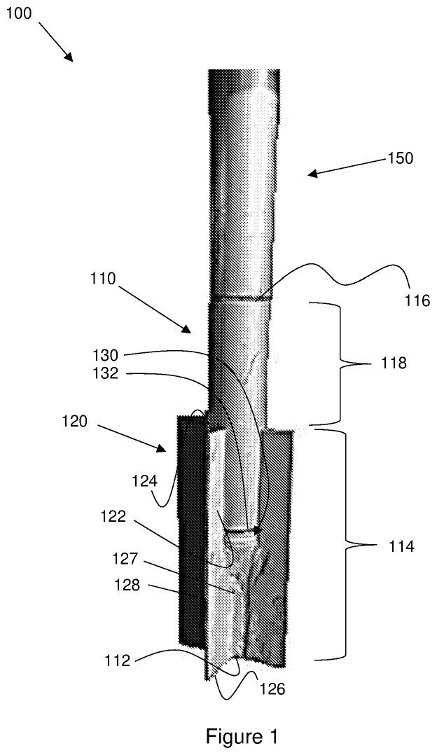

FIGS. 1 to 4 show embodiments of a device (100) for simultaneously measuring shear strength and pore water pressure of a soil in the field when performing a field vane shear test.

Referring to FIG. 1, the device (100) includes a rod (110) adapted to be at least partially inserted into a soil of an earthen substrate. The rod (110) has a soil engaging portion (114) located at or near a soil engaging end (112) and an opposed coupling portion (118) located at or near a torque applying machine or device engaging end (116).

The device (100) also includes four vane blades (120) extending at least partially along and from the soil engaging portion (114) for shearing soil when rotated together with the rod (110).

The device (100) further includes a pressure sensor (130) located on the soil engaging portion (114) at least partially between the vane blades (120) for sensing pressure indicative of pore water pressure of the soil while the vane blades (120) shear the soil.

Generally, the soil may form part of an earthen substrate. For example, the earthen substrate may include a portion of land, an embankment, a levee, a transportation embankment, an embankment dam, an earthen dam wall (e.g., a tailings dam wall) or reclaimed land alongside a body of water, such as, e.g., a riverbank or beach. The soil may include any non-fissured clay, silt or other saturated fine-grained geomaterials, such as, e.g., mine tailings, organic muck, slimes, leach residues, slickens, dredge spoils and other like material and/or materials.

The rod (110) is formed of stainless steel.

As indicated, the rod (110) includes a pair of opposed ends (112, 116) and extends longitudinally between the opposed ends (112, 116) in a linear direction.

The coupling portion (118), located at or near the torque applying machine or device engaging end (116), is for coupling to a torque applying machine or device.

The coupling portion (118) can couple directly, or indirectly via a torque rod (150) as shown, with the torque applying machine or device.

Generally, the torque applying machine or device includes an actuating mechanism for rotation of the rod (110) in a clockwise or anti-clockwise direction. The actuating mechanism may typically be driven by a drive motor.

The coupling portion (118) and the torque applying machine or device may be connected together by a connecting mechanism or part of a connecting mechanism, a clamping mechanism or a socket type connection.

The vane blades (120) are also formed of stainless steel. Each blade (120) has a rectangular shape defined by a pair of opposed side surfaces (122), an upper edge (124), an opposed lower edge (126), an inner side edge (127) and an opposed outer side edge (128).

The rod (110) and the vane blades (120) are joined together using conventional welding techniques.

As shown, each vane blade (120) extends from the rod (110) in a direction perpendicular to a longitudinal axis of the rod (110).

The vane blades (120) are arranged in diametrically opposed pairs about the soil engaging portion (114) of the rod (110).

Each blade (120) typically has a height, measured as the distance that the blade (120) extends at least partially along the soil engaging portion (114) of the rod (110), that is approximately two times the diameter of a diametrically opposed pair of the blades (120).

Each blade (120) has a thickness ranging between about 1.5 mm and about 3.5 mm.

As mentioned, the device (100) includes a pressure sensor (130) located on the soil engaging portion (114) at least partially between the vane blades (120).

The pressure sensor (130) is a piezoelectric sensor and is provided in a casing including a cavity and a groundwater inlet port (132). The pressure sensor (130) measures the pressure of groundwater that passes through the groundwater inlet port (132) into the cavity, the pressure being indicative of the pore water pressure.

The groundwater inlet port (132) includes an opening of suitable size to allow the passage of groundwater and a filter to prevent the passage of soil materials other than groundwater into the cavity via the opening. The filter has a pore size of between about 2 μm and about 20 μm.

The filter is removable and/or replaceable so that the filter can be cleaned and re-fitted or replaced between field vane tests or as required.

The pressure sensor (130) is electrically connected to a data bus or like connection.

The device (100) includes a dedicated microprocessor or microcomputer, including one or more processors and a memory, operatively associated with the pressure sensor (130) for collecting data corresponding to the pressure sensed and transmitting the data to an external device, controller or processing device. The data is at least partially transmitted via at least one electrical circuit extending along and within the rod (110), or portions thereof.

In some embodiments, the device (100) further includes a communications module for wirelessly connecting the device to a remote controller in the form of an external processing device via a wireless network (e.g., Wi-Fi (WLAN) communication, Satellite communication, RF communication, infrared communication or Bluetooth™). The communications module includes a cellular or radio modem.

The external processing device can be any one of a desktop, a laptop, a smart phone, a tablet or smart watch. The external processing device includes software configured to run on the external processing device and allow an operator to control at least aspects of operation of the device (100) and display and analyse data transmitted from the device (100).

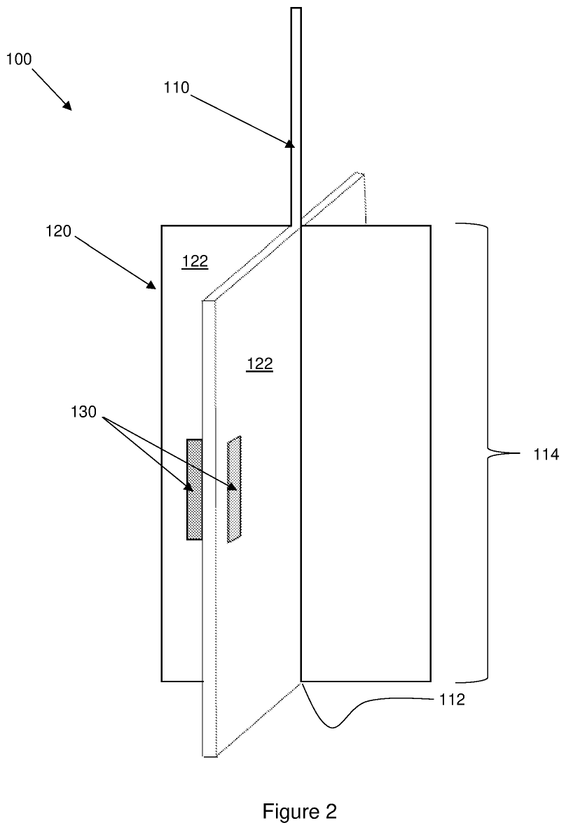

FIG. 2 shows another embodiment of the device (100). For convenience, features that are similar or correspond to features of the previous embodiment will be referenced with the same reference numerals.

Referring to FIG. 2, the device (100) again includes a rod (110) having a soil engaging portion (114) located at or near a soil engaging end (112) and four vane blades (120) extending therefrom.

However, in contrast to the previous embodiment, in this embodiment the device (100) includes a plurality of pressure sensors (130) each located on a side surface (122) of a vane blade (120).

In a variation of this embodiment, the device (100) can include pressure sensors (130) located on both opposed side surfaces (122) of each blade (120).

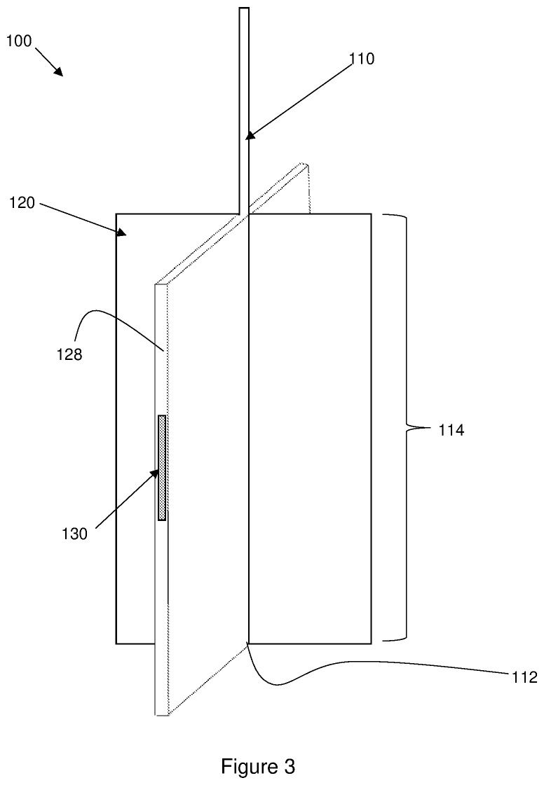

FIG. 3 shows another embodiment of the device (100). Again, and for convenience, features that are similar or correspond to features of the previous embodiment will be referenced with the same reference numerals.

Referring to FIG. 3, the device (100) again includes a rod (110) having a soil engaging portion (114) located at or near a soil engaging end (112) and four vane blades (120) extending therefrom.

However, in contrast to the previous embodiments, in this embodiment the device (100) includes a plurality of pressure sensors (130) each located on an outer side edge (128) of each blade (120).

In a variation of this embodiment, the device (100) can include pressure sensors (130) on only one pair of the vane blades (120).

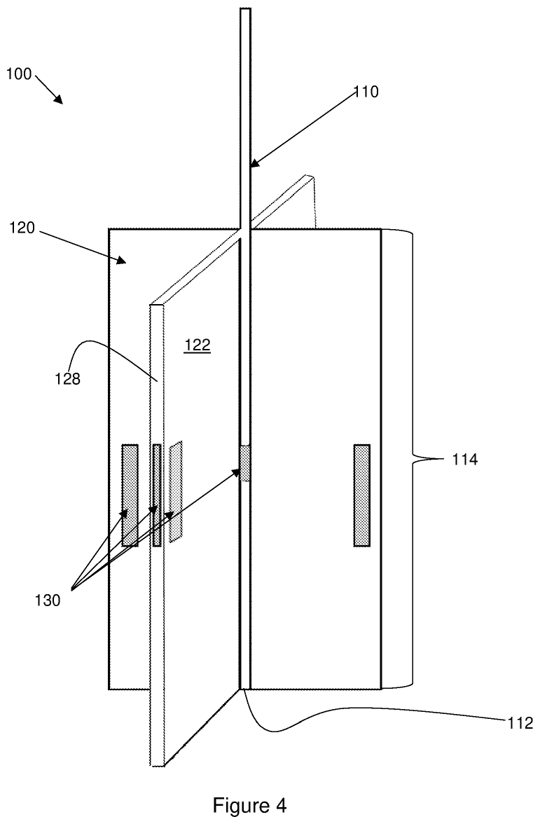

FIG. 4 shows another embodiment of the device (100). Again, and for convenience, features that are similar or correspond to features of the previous embodiment will be referenced with the same reference numerals.

Referring to FIG. 4, the device (100) again includes a rod (110) having a soil engaging portion (114) located at or near a soil engaging end (112) and four vane blades (120) extending therefrom.

However, in contrast to the previous embodiments, in this embodiment the device (100) includes a plurality of pressure sensors (130) located on both side surfaces (122) and the outer side edge (128) of each vane blade (120) as well as on the soil engaging portion (114) at least partially between the vane blades (120).

In a variation of this embodiment, the device (100) can include pressure sensors (130) on only one pair of the vane blades (120).

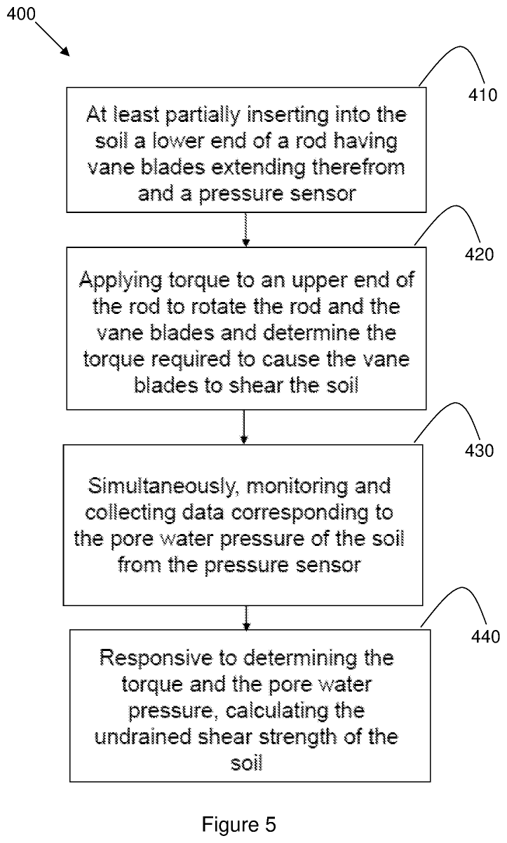

A method (400) of using the device (100) as shown in any one of FIGS. 1 to 4 will now be described in detail with reference to FIG. 5.

At step 410, the soil engaging portion (114) of the rod (110) is at least partially inserted into a soil layer of interest such that the vane blades (120) fully penetrate the intact soil layer.

At step 420, torque is applied to the coupling portion (118) of the rod (110) such that the rod (110) and vane blades (120) rotate at a constant rate. The torque is increased until the vane blades (120) cause the soil to shear. The torque required to cause the vane blades (120) to shear the soil is noted and recorded.

At step 430, while applying torque, data corresponding to the pore water pressure of the groundwater in the soil layer is monitored and collected via the pressure sensor (130). The monitoring and collecting occurs in real time.

At step 440, the undrained shear strength of the soil layer is determined based on the torque reached at step 420. The undrained shear strength is adjusted to account for the effect of the pore water pressure measured at step 430. For example, the pore water pressure can be accounted for and/or subtracted from the torque determined at step 420 when determining the undrained shear strength of the soil layer to provide a more accurate measurement of the shear strength of the soil.

Any of the features described herein can be combined in any combination with any one or more of the other features described herein within the scope of the invention.

### EXAMPLE

The following example is provided to demonstrate the effectiveness of the device (100) of the present invention. The example is not to be considered limiting on the scope and ambit of the present invention as hereinbefore described.

### Example 1

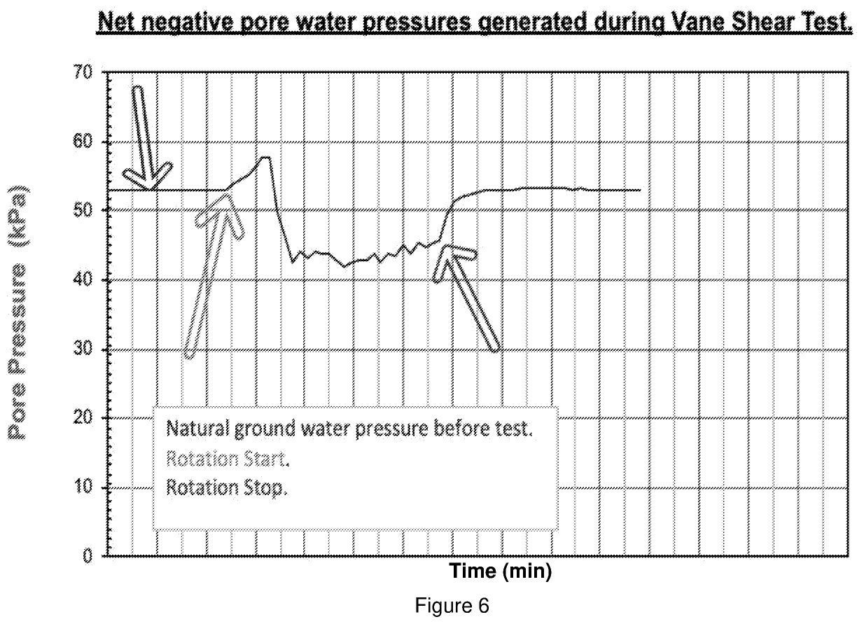

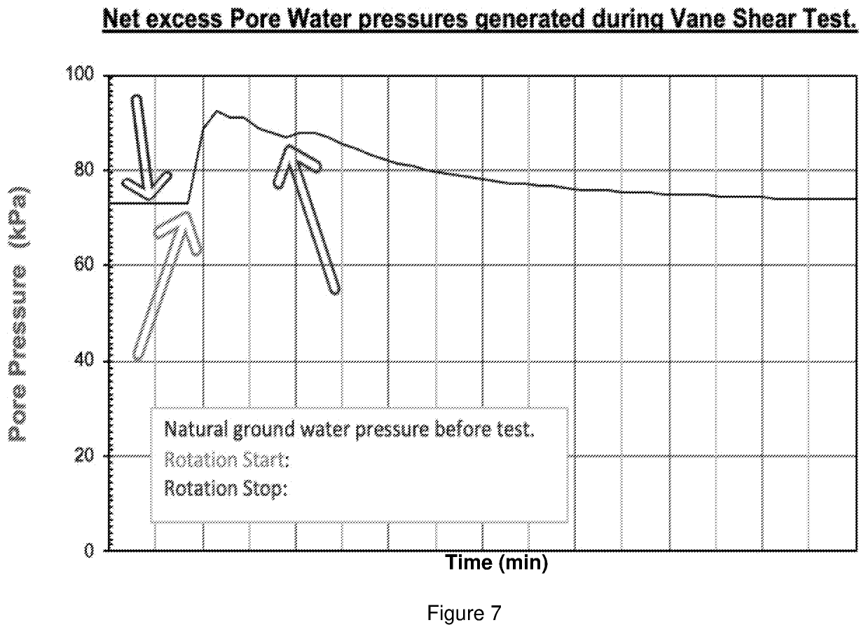

Referring to FIGS. 6 and 7, field vane shear tests were conducted on privately owned land in May 2020.

The tests were conducted using the device (100) as shown in FIG. 1 coupled to a piezocone (CPTu). The tests were conducted on an earthen substrate consisting of mine tailing at locations approximately 100 m from one another.

Referring to FIG. 6, in this test, the pore water pressure can be seen to be stable and hydrostatic before commencement of the field vane shear test. However, once the device (100) starts to rotate and increasing amounts of torque are applied, the pore water pressure initially spikes before dropping for the remainder of the test. When the test concludes, the pore water pressure once again stabilises to a level measured before initiation of the test.

These test results suggest that the soil is dilative and the pore water is initially compressed between the soil particles when the test initiates causing the pore water pressure to initially spike before the pore water drains away from the site of the test.

The measurement of the pore water pressure during the test enables the pore water pressure to be accounted for when determining the undrained shear strength of the soil. If the pore water pressure were not measured, it is likely that the undrained shear strength of the soil would be incorrectly calculated.

Referring to FIG. 7, in this test, the pore water pressure can be seen to again be stable and hydrostatic before commencement of the field vane shear test. However, once the device (100) starts to rotate and increasing amounts of torque are applied, the pore water pressure spikes and remains elevated for the duration of the test. When the test concludes, the pore water pressure gradually stabilises to a level measured before initiation of the test.

These test results suggest that the soil has a low hydraulic conductivity with rotation of the device (100) causing the pore water between soil particles to compress and generate an artificial spike in pore water pressure for a duration of the test.

The measurement of the pore water pressure during this test again enables the pore water pressure to be accounted for when determining the undrained shear strength of the soil. If the pore water pressure were not measured, it is likely that the undrained shear strength of the soil would be incorrectly calculated.

In the present specification and claims (if any), the word ‘comprising’ and its derivatives including ‘comprises’ and ‘comprise’ include each of the stated integers but does not exclude the inclusion of one or more further integers.

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristics may be combined in any suitable manner in one or more combinations.

In compliance with the statute, the invention has been described in language more or less specific to structural or methodical features. It is to be understood that the invention is not limited to specific features shown or described since the means herein described comprises preferred forms of putting the invention into effect. The invention is, therefore, claimed in any of its forms or modifications within the proper scope of the appended claims (if any) appropriately interpreted by those skilled in the art.

1. A device for selectively or simultaneously measuring shear strength and pore water pressure of a soil in the field, said device comprising:
a rod adapted to be at least partially inserted into the soil and rotated, said rod having a soil engaging portion and an opposed coupling portion for coupling to a toque applying machine or device;
at least one vane blade extending at least partially along and from the soil engaging portion of the rod for shearing the soil when rotated together with the rod, said at least one vane blade having a leading face, an opposed trailing face, an upper edge, an opposed lower edge and an outer side edge; and
a plurality of pore water pressure sensors operatively associated with two or more of the soil engaging portion, the leading face, the trailing face and the outer side edge, said sensors configured to collectively sense pressure indicative of the pore water pressure of the soil while the at least one vane blade shears the soil.

2. The device of claim 1, wherein the at least one vane blade includes four vane blades extending along and from the soil engaging portion of the rod in an opposed arrangement.

3. The device of claim 2, wherein the plurality of pore water pressure sensors are located on the outer side edge and the leading face of the at least one vane blade.

4. The device of claim 3, wherein the plurality of pore water pressure sensors further comprise a pore water pressure sensor located on the soil engaging portion of the rod.

5. The device of claim 4, wherein the plurality of pore water pressure sensors further comprise a pore water pressure sensor located on the trailing face of at least one vane blade.

6. The device of claim 5, further comprising a plurality of casings each having at least one ground water inlet port and configured to contain one of the plurality of pore water pressure sensors, each said casing configured to enable a flow of groundwater into the inlet port and pressure indicative of the pore water pressure to be measured.

7. The device of claim 6, wherein the at least one ground water inlet port of each casing further comprises a screen or filter to prevent the passage of materials other than groundwater into the inlet port.

8. The device of claim 7, wherein the screen or filter has a pore size of between about 2 μm and about 20 μm.

9. The device of claim 7, wherein the screen or filter is removable and replaceable so that the screen or filter can be cleaned or replaced between measurements or as required.

10. The device of claim 6, wherein each said casing is filled with a fluid having a viscosity greater than water and wherein pressure indicative of the pore water pressure is measured based on the pressure of the ground water on the fluid in the casing.

11. The device of claim 10, wherein the fluid is selected from any one of glycerin, silicone oil and grease.

12. The device of claim 11, wherein the fluid is added and replaced between measurements with the device or as required.

13. The device of claim 10, further comprising a cleaning mechanism for cleaning or flushing the pore water pressure sensors.

14. The device of claim 13, wherein each said pore water pressure sensor is operatively associated with a source of the fluid for flushing the sensor following or in between measurements with the device.

15. The device of claim 14, wherein the fluid is delivered from a fluid source into the casing of each pore water pressure sensor via one or more outlets in fluid communication with the casing and the source.

16. A system for selectively or simultaneously measuring shear strength and pore water pressure of a soil in the field, said system comprising:
a device in accordance with claim 1; and
a torque applying machine having a controller coupled to the coupling portion of the rod of the device for rotating the rod, said controller configured to control rotation of the rod at a desired rate of rotation.

17. The system of claim 16, wherein the controller further controls an angle of rotation and an amount of torque applied to the rod.

18. The system of claim 16, wherein the desired rate of rotation ranges from about 0.05° per second to about 1.00° per second.

19. A method of selectively or simultaneously measuring shear strength and pore water pressure of a soil in the field, said method comprising:
providing the device of claim 1;
at least partially inserting the soil engaging portion and the at least one vane blade into the soil;
applying torque to the coupling portion to rotate the rod and the at least one vane blade to cause the at least one vane blade to shear the soil; and
monitoring and collecting at least data corresponding to pore water pressure from the at least one pressure sensor simultaneously with said applying.

20. The method of claim 19, wherein the monitoring and collecting occur in real time and continuously while the torque is applied to rotate the rod.
